(19) United States
(12) Patent Application Publication
(10) Pub. No.: US 2024/0389844 A1
(43) Pub. Date: Nov. 28, 2024

(54) DENTAL IMAGING SYSTEM, AND DENTAL ROBOT SYSTEM INCLUDING SAME

(71) Applicant: NEOCIS INC., Miami, FL (US)

(72) Inventors: Alon MOZES, Miami Beach, FL (US); Dennis MOSES, Hollywood, FL (US); Erik Paul Flor BEUCKELAERS, San Diego, CA (US)

(21) Appl. No.: 18/692,460

(22) PCT Filed: Sep. 16, 2022
(86) PCT No.: PCT/IB2022/058772

§ 371 Date: Mar. 15, 2024

Related U.S. Application Data
(60) Provisional application No. 63/245,306, filed on Sep. 17, 2021.

(51) Int. Cl. A61B 1/24; A61B 1/00; A61B 1/07; A61B 34/20; A61B 34/30

(52) U.S. Cl. CPC A61B 1/24; A61B 1/00002; A61B 1/00048; A61B 1/0005; A61B 1/07; A61B 34/20; A61B 34/30; A61B 2034/302

(57) ABSTRACT

A dental imaging system includes a dental tool having an end effector adapted to interact with an object. An optical imaging device is engaged with the dental tool or the end effector, and arranged such that a field-of-view thereof includes the interaction between the end effector and the object. A display is in communication with the optical imaging device and is arranged to display a real-time image of the interaction between the end effector and the object received from the optical imaging device. Associated dental robotic systems implementing the dental imaging system are also provided.

### BACKGROUND

### Field of the Disclosure

The present disclosure relates to dental robot systems and, more particularly to a dental imaging system and dental robot system incorporating such a dental imaging system.

### Description of Related Art

Illnesses are commonly shared in the workplace, but the risk of exposure may be significantly higher for dental professionals using powered instrumentation such as ultrasonics, air polishers, and drills. Bacteria and viruses can spread rapidly through splatter, aerosols, and particulate debris produced by these types of treatments. That is, aerosols, particulate debris, and splatter produced, for example, by ultrasonic, drilling, and air polishing treatments can contain saliva, blood, bacteria, and pathogens. Once airborne, aerosol particles can linger in the environment for an hour or more while particulate debris and splatter can end up on the surfaces immediately surrounding the treatment area. This poses a risk, for example, for the spread of the common cold and influenza viruses, herpes viruses, pathogenic streptococci or staphylococci, severe acute respiratory syndrome (SARS), and tuberculosis (TB), with primary risk to the dental professional.

Moreover, in conducting dental procedures using such powered instrumentation, the patient is often in a reclined position, and the dental professional must contort and get close to the patient's mouth, or maneuver a small handheld mirror, in order to be able to view the procedure as it is being conducted. Accordingly, such dental procedures can often be cumbersome, non-user-friendly, and non-ergonomic for the dental professional.

Thus, there exists a need for a dental system for facilitating effective regulation of the spread of contagions and contaminants, particularly to the dental professional, by limiting the spread of dental aerosols and particulate debris resulting from a maxillofacial procedure. There also exists a need for a dental system that would allow the dental professional to conduct and view a maxillofacial procedure in a user-friendly, distanced, and ergonomic manner. Such a system should preferably be effective without limiting the mobility of the dental tool, the accessibility of the dental tool to the maxillofacial structure, or the ability of the dental professional to see the maxillofacial structure without having to be too close to the actual interaction between the dental tool and the maxillofacial structure. Moreover, such a system should be ergonomically friendly for the dental professional, for example, by allowing one-handed operation so as to allow the dental professional to use other instruments (e.g., a dental mirror or suction device) concurrently with using the system.

### SUMMARY

The above and other needs are met by aspects of the present disclosure which, in one aspect, provides a dental imaging system, comprising a dental tool having an end effector adapted to interact with an object. An optical imaging device is engaged with the dental tool or the end effector, and arranged such that a field-of-view thereof includes the interaction between the end effector and the object. A display is in communication with the optical imaging device and arranged to display a real-time image of the interaction between the end effector and the object received from the optical imaging device.

Another aspect of the present disclosure provides a dental robotic system, comprising a fiducial marker adapted to engage an object. An articulating arm has a dental tool operably engaged with a distal end thereof, and the dental tool has an end effector adapted to interact with the object. An optical imaging device is engaged with the dental tool or the end effector, and is arranged such that a field-of-view thereof includes an interaction between the end effector and the object. A controller is arranged in communication with the articulating arm, the dental tool, and the fiducial marker. The controller is arranged to determine a disposition of the end effector in relation to the fiducial marker during movement of the end effector to interact with the object, and to direct the articulating arm to physically control allowable movement of the dental tool, directly relative to the disposition of the end effector with respect to the fiducial marker engaged with the object. A display is in communication with the optical imaging device and is arranged to display a real-time image of the interaction between the end effector and the object received from the optical imaging device.

Yet another aspect of the present disclosure provides a dental robotic system, comprising an articulating arm having a proximal end and a distal end opposed thereto. One or more sensors is operably engaged with the articulating arm and arranged to sense position data associated with the articulating arm. A dental tool is operably engaged with a distal end of the articulating arm and has an end effector adapted to interact with an object. An optical imaging device is engaged with the distal end of the articulating arm, the dental tool, or the end effector, in known relation to the end effector, and is arranged such that a field-of-view thereof includes an interaction between the end effector and the object. The optical imaging device is further arranged to capture imaging data. A controller is arranged in communication with the articulating arm, the one or more sensors, the dental tool, and the optical imaging device. The controller is arranged to receive the position data associated with the articulating arm from the one or more sensors, the position data indicating a disposition of the optical imaging device in relation to the proximal end of the articulating arm, and to receive the imaging data associated with an image of the object captured by the optical imaging device, the imaging data indicating a disposition of the object in relation to the optical imaging device. The controller is further arranged to determine a disposition of the end effector in relation to the object, from the position data associated with the articulating arm and the imaging data associated with the image, during movement of the end effector to interact with the object, and to direct the articulating arm member to physically control allowable movement of the dental tool, directly relative to the disposition of the end effector with respect to the object. A display is in communication with the optical imaging device and is arranged to display a real-time image of the interaction between the end effector and the object received from the optical imaging device.

The present disclosure thus includes, without limitation, the following embodiments:

Embodiment 1: A dental imaging system, comprising a dental tool having an end effector adapted to interact with an object; an optical imaging device engaged with the dental tool or the end effector, and arranged such that a field-of-view thereof includes the interaction between the end effector and the object; and a display in communication with the optical imaging device and arranged to display a real-time image of the interaction between the end effector and the object received from the optical imaging device.

Embodiment 2: The system of any preceding embodiment, or any combination of preceding embodiments, wherein the dental tool is a drill and the end effector is a drill bit or an abrading bit, wherein the dental tool is an ultrasonic cleaner and the end effector is a cleaning tip, or wherein the dental tool is a pneumatic polisher and the end effector is a polishing tip.

Embodiment 3: The system of any preceding embodiment, or any combination of preceding embodiments, wherein the display is mounted on the dental tool, is disposed remotely to the dental tool, or is mounted within a headset adapted to be worn by a user.

Embodiment 4: The system of any preceding embodiment, or any combination of preceding embodiments, wherein the dental tool is engaged with an articulating arm of a dental robotic system.

Embodiment 5: The system of any preceding embodiment, or any combination of preceding embodiments, wherein the optical imaging device includes a camera, an imaging array, or an optical fiber disposed adjacent the end effector of the dental tool.

Embodiment 6: The system of any preceding embodiment, or any combination of preceding embodiments, wherein the end effector defines an axial channel extending toward a distal end of the dental tool, and wherein the optical fiber is disposed within and extends along the axial channel such that a distal end of the optical fiber is disposed proximate the distal end of the dental tool.

Embodiment 7: The system of any preceding embodiment, or any combination of preceding embodiments, comprising a light-emitting device arranged to illuminate the object or the end effector.

Embodiment 8: The system of any preceding embodiment, or any combination of preceding embodiments, wherein the optical imaging device is arranged to automatically focus within the field-of-view.

Embodiment 9: The system of any preceding embodiment, or any combination of preceding embodiments, wherein the optical imaging device is arranged in communication with the display via a wireless communication system.

Embodiment 10: The system of any preceding embodiment, or any combination of preceding embodiments, wherein the display is arranged to display the real-time image of the interaction between the end effector and the object combined with an image of the object not obtained from the optical imaging device, so as to form an augmented virtual representation of the interaction.

Embodiment 11: A dental robotic system, comprising a fiducial marker adapted to engage an object; an articulating arm having a dental tool operably engaged with a distal end thereof and having an end effector adapted to interact with the object, and an optical imaging device engaged with the dental tool or the end effector, and arranged such that a field-of-view thereof includes an interaction between the end effector and the object; a controller arranged in communication with the articulating arm, the dental tool, and the fiducial marker, the controller being arranged to determine a disposition of the end effector in relation to the fiducial marker during movement of the end effector to interact with the object, and direct the articulating arm to physically control allowable movement of the dental tool, directly relative to the disposition of the end effector with respect to the fiducial marker engaged with the object; and a display in communication with the optical imaging device and arranged to display a real-time image of the interaction between the end effector and the object received from the optical imaging device.

Embodiment 12: The system of any preceding embodiment, or any combination of preceding embodiments, wherein the dental tool is a drill and the end effector is a drill bit or an abrading bit, wherein the dental tool is an ultrasonic cleaner and the end effector is a cleaning tip, or wherein the dental tool is a pneumatic polisher and the end effector is a polishing tip.

Embodiment 13: The system of any preceding embodiment, or any combination of preceding embodiments, wherein the display is mounted on the dental tool, is disposed remotely to the dental tool, or is mounted within a headset adapted to be worn by a user.

Embodiment 14: The system of any preceding embodiment, or any combination of preceding embodiments, wherein the optical imaging device includes a camera, an imaging array, or an optical fiber disposed adjacent the end effector of the dental tool.

Embodiment 15: The system of any preceding embodiment, or any combination of preceding embodiments, wherein the end effector defines an axial channel extending to a distal end of the dental tool, and wherein the optical fiber is disposed within and extends along the axial channel such that a distal end of the optical fiber is disposed proximate the distal end of the dental tool.

Embodiment 16: The system of any preceding embodiment, or any combination of preceding embodiments, comprising a light-emitting device arranged to illuminate the object or the end effector.

Embodiment 17: The system of any preceding embodiment, or any combination of preceding embodiments, wherein the optical imaging device is arranged to automatically focus within the field-of-view.

Embodiment 18: The system of any preceding embodiment, or any combination of preceding embodiments, further comprising a detector engaged with a distal end of a tracking arm, the tracking arm and the detector being in communication with the controller, the detector being arranged in a spaced-apart relationship with the fiducial marker to detect the fiducial marker and to cooperate with the controller to determine a spatial relationship between the optical imaging device and the fiducial marker or between the end effector and the fiducial marker.

Embodiment 19: The system of any preceding embodiment, or any combination of preceding embodiments, wherein the detector is an electrical detector, an electromechanical detector, an electromagnetic detector, an optical detector, an infrared detector, or combinations thereof.

Embodiment 20: The system of any preceding embodiment, or any combination of preceding embodiments, comprising a tracking arm having a distal end physically engaged with the fiducial marker, the tracking arm being in communication with the controller and arranged to cooperate with the controller to determine a spatial relationship between the optical imaging device and the fiducial marker or between the end effector and the fiducial marker.

Embodiment 21: The system of any preceding embodiment, or any combination of preceding embodiments, wherein the optical imaging device is arranged in communication with the display via a wireless communication system.

Embodiment 22: The system of any preceding embodiment, or any combination of preceding embodiments, wherein the display is arranged to display the real-time image of the interaction between the end effector and the object combined with an image of the object not obtained from the optical imaging device, so as to form an augmented virtual representation of the interaction.

Embodiment 23: A dental robotic system, comprising an articulating arm having a proximal end and a distal end opposed thereto; one or more sensors operably engaged with the articulating arm and arranged to sense position data associated with the articulating arm; a dental tool operably engaged with the distal end of the articulating arm and having an end effector adapted to interact with an object; an optical imaging device engaged with the distal end of the articulating arm, the dental tool, or the end effector, in known relation to the end effector and arranged such that a field-of-view thereof includes an interaction between the end effector and the object, the optical imaging device being further arranged to capture imaging data; a controller arranged in communication with the articulating arm, the one or more sensors, the dental tool, and the optical imaging device, the controller being arranged to: receive the position data associated with the articulating arm from the one or more sensors, the position data indicating a disposition of the optical imaging device in relation to the proximal end of the articulating arm, receive the imaging data associated with an image of the object captured by the optical imaging device, the imaging data indicating a disposition of the object in relation to the optical imaging device, determine a disposition of the end effector in relation to the object, from the position data associated with the articulating arm and the imaging data associated with the image, during movement of the end effector to interact with the object, and direct the articulating arm to physically control allowable movement of the dental tool, directly relative to the disposition of the end effector with respect to the object; and a display in communication with the optical imaging device and arranged to display a real-time image of the interaction between the end effector and the object received from the optical imaging device.

Embodiment 24: The system of any preceding embodiment, or any combination of preceding embodiments, wherein the dental tool is a drill and the end effector is a drill bit or an abrading bit, wherein the dental tool is an ultrasonic cleaner and the end effector is a cleaning tip, or wherein the dental tool is a pneumatic polisher and the end effector is a polishing tip.

Embodiment 25: The system of any preceding embodiment, or any combination of preceding embodiments, wherein the display is mounted on the dental tool, is disposed remotely to the dental tool, or is mounted within a headset adapted to be worn by a user.

Embodiment 26: The system of any preceding embodiment, or any combination of preceding embodiments, wherein the optical imaging device includes a camera, an imaging array, or an optical fiber disposed adjacent the end effector of the dental tool.

Embodiment 27: The system of any preceding embodiment, or any combination of preceding embodiments, wherein the end effector defines an axial channel extending toward a distal end of the dental tool, and wherein the optical fiber is disposed within and extends along the axial channel such that a distal end of the optical fiber is disposed proximate the distal end of the dental tool.

Embodiment 28: The system of any preceding embodiment, or any combination of preceding embodiments, comprising a light-emitting device arranged to illuminate the maxillofacial structure or the end effector.

Embodiment 29: The system of any preceding embodiment, or any combination of preceding embodiments, wherein the optical imaging device is arranged to automatically focus within the field-of-view.

Embodiment 30: The system of any preceding embodiment, or any combination of preceding embodiments, wherein the optical imaging device is arranged in communication with the display via a wireless communication system.

Embodiment 31: The system of any preceding embodiment, or any combination of preceding embodiments, wherein the display is arranged to display the real-time image of the interaction between the end effector and the object combined with an image of the object not obtained from the optical imaging device, so as to form an augmented virtual representation of the interaction.

Embodiment 32: The system of any preceding embodiment, or any combination of preceding embodiments, wherein the controller is arranged to associate the position data associated with the articulating arm from the one or more sensors, and the imaging data associated with each of a plurality of two-dimensional images of the object or the interaction between the end effector and the object captured by the optical imaging device.

Embodiment 33: The system of any preceding embodiment, or any combination of preceding embodiments, wherein the controller is arranged to combine together the plurality of two-dimensional images of the object or the interaction between the end effector and the object, based on the position data and the imaging data, and to form a three-dimensional augmented virtual representation of the object or the interaction between the end effector and the object.

Embodiment 34: The system of any preceding embodiment, or any combination of preceding embodiments, further comprising a detector engaged with a distal end of a tracking arm, the tracking arm and the detector being in communication with the controller, the detector being arranged in a spaced-apart relationship with the fiducial marker to detect the fiducial marker and to cooperate with the controller to determine a spatial relationship between the optical imaging device and the fiducial marker or between the end effector and the fiducial maker, or further comprising a tracking arm having a distal end physically engaged with the fiducial marker, the tracking arm being in communication with the controller and arranged to cooperate with the controller to determine the spatial relationship between the optical imaging device and the fiducial marker or between the end effector and the fiducial marker.

Embodiment 35: The system of any preceding embodiment, or any combination of preceding embodiments, wherein the detector is an electrical detector, an electromechanical detector, an electromagnetic detector, an optical detector, an infrared detector, or combinations thereof.

Embodiment 36: The system of any preceding embodiment, or any combination of preceding embodiments, wherein the controller is arranged to associate the position data associated with the articulating arm from the one or more sensors, the imaging data associated with an image of the object captured by the optical imaging device, and the spatial relationship between the optical imaging device and the fiducial marker with each of a plurality of two-dimensional images of the object or the interaction between the end effector and the object captured by the optical imaging device.

Embodiment 37: The system of any preceding embodiment, or any combination of preceding embodiments, wherein the controller is arranged to combine the plurality of two-dimensional images of the object or the interaction between the end effector and the object together, based on the position data, the imaging data, and the spatial relationship between the optical imaging device and the fiducial marker, and form a three-dimensional augmented virtual representation of the object or the interaction of the end effector with the object.

These and other features, aspects, and advantages of the present disclosure will be apparent from a reading of the following detailed description together with the accompanying drawings, which are briefly described below. The present disclosure includes any combination of two, three, four, or more features or elements set forth in this disclosure, regardless of whether such features or elements are expressly combined or otherwise recited in a specific embodiment description herein. This disclosure is intended to be read holistically such that any separable features or elements of the disclosure, in any of its aspects and embodiments, should be viewed as intended, namely to be combinable, unless the context of the disclosure clearly dictates otherwise.

It will be appreciated that the summary herein is provided merely for purposes of summarizing some example aspects so as to provide a basic understanding of the disclosure. As such, it will be appreciated that the above described example aspects are merely examples and should not be construed to narrow the scope or spirit of the disclosure in any way. It will be appreciated that the scope of the disclosure encompasses many potential aspects, some of which will be further described below, in addition to those herein summarized. Further, other aspects and advantages of such aspects disclosed herein will become apparent from the following detailed description taken in conjunction with the accompanying drawings which illustrate, by way of example, the principles of the described aspects.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIG. 1 schematically illustrates a dental imaging system, according to one aspect of the present disclosure;

FIG. 2 schematically illustrates a dental imaging system, according to another aspect of the present disclosure:

FIG. 3 schematically illustrates a dental imaging system, according to one aspect of the present disclosure, interfaced with one aspect of a dental robotic system:

FIG. 4 schematically illustrates a dental imaging system, according to one aspect of the present disclosure, interfaced with another aspect of a dental robotic system; and

FIG. 5 schematically illustrates a dental imaging system, according to one aspect of the present disclosure, interfaced with yet another aspect of a dental robotic system.

### DETAILED DESCRIPTION OF THE DISCLOSURE

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein; rather, these aspects are provided so that this disclosure will satisfy applicable legal requirements. Like numbers refer to like elements throughout.

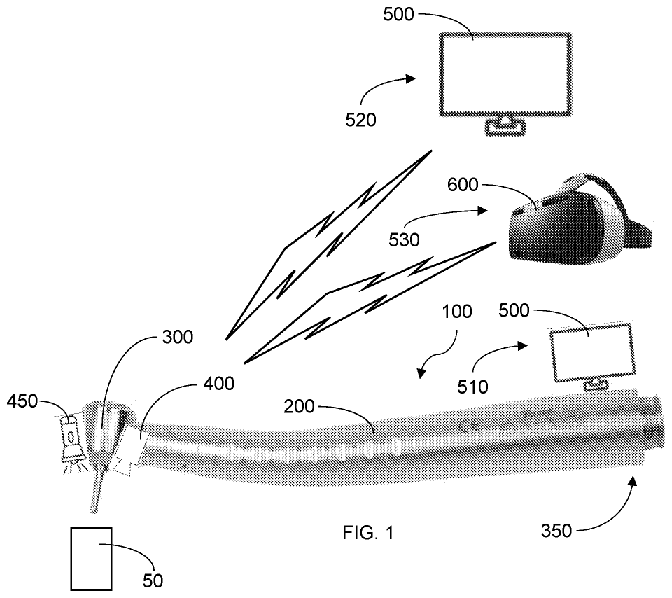

FIG. 1 schematically illustrates a dental imaging system 100 according to one aspect of the present disclosure. Such a system includes a dental tool 200 having an end effector 300 adapted to interact with an object 50 such as a maxillofacial structure. In some aspects, as shown for example in FIG. 1, the dental tool 200 is a drill and the end effector 300 is a drill bit or an abrading bit. In other aspects, the dental tool 200 is an ultrasonic cleaner and the end effector 300 is a cleaning tip. In yet other aspects, the dental tool 200 is a pneumatic polisher and the end effector 300 is a polishing tip.

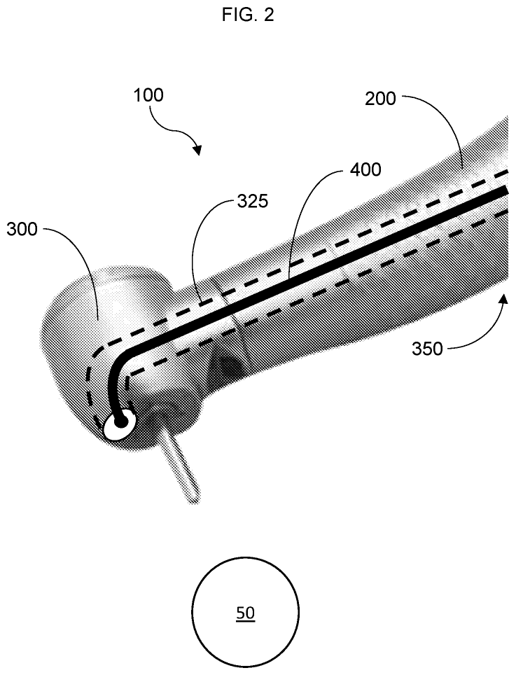

In particular aspects, an optical imaging device 400 is engaged with the dental tool 200 or the end effector 300. The optical imaging device 400 is arranged with respect to the dental tool 200 or end effector 300 such that a field-of-view of the optical imaging device 400 includes at least the interaction between end effector 300 and the object 50 (e.g., maxillofacial structure). The system 100 can also include a light-emitting device 450 arranged to illuminate the object and/or the end effector 300. The optical imaging device 400 is, for example, a camera, an imaging array, or an optical fiber, disposed adjacent the end effector 300 of the dental tool 200, and optionally arranged to automatically focus within the field-of-view (e.g., automatically focus on the object). The optical imaging device 400, in its various forms, can be further arranged to capture individual still or two-dimensional images or, in other aspects, can be arranged to capture video (e.g., continuous imaging or real time video) of the object 50 or the interaction between the end effector 300 and the object 50. In some aspects, with the optical imaging device 400 in the form of an optical fiber (see, e.g., FIG. 2 or where the camera or imaging array is a wired device), the end effector 300 defines an axial channel 325 extending toward a distal end 350 of the dental tool 200. In such instances, the optical fiber is disposed within and extends along the axial channel 325 such that a distal end of the optical fiber is disposed proximate the distal end 350 of the dental tool 200. In other instances, a connecting wire associated with the camera or imaging array can be disposed within the axial channel 325 and extend therealong to connect to the camera of imaging array connected to or associated with the end effector 300.

In some aspects, a display 500 (see, e.g., FIG. 1) is in communication with the optical imaging device 400, wherein the display 500 is configured/arranged to display a real-time image of the interaction between the end effector 300 and the object 50 (e.g., maxillofacial structure), received from the optical imaging device 400. In some particular instances, the display 500 is mounted on the dental tool 200 (see, e.g., 510 in FIG. 1), is disposed remotely to the dental tool 200 (see, e.g., 520 in FIG. 1), or is mounted within a headset 600 adapted to be worn by a user (see, e.g., 530 in FIG. 1). The optical imaging device 400 and display 500 are arranged in communication with each other, for example, via a wireless communication system though, in some instances, the communication can be accomplished by way of a wired communication system.

During the interaction between the end effector 300 and the object 50, certain aerosols can be formed from the interaction such as, for example, from particles of the object 50 or water, saliva, and/or blood associated with the maxillofacial structure. In addition to the aerosol(s), certain particulate debris can also become airborne, where such particulate debris can include, for example, object particles, plaque/tartar, tooth/bone particles, and/or food particles. The aerosol(s) and/or particulate debris can be directed outwardly of the object 50 (e.g., along the direction between the end effector 300 and a distal end 350 of the dental tool 200). Mounting the display 500 in one of the disclosed manners allows the user to view, in real time, the interaction between the object 50 and the end effector 300, while the user is removed or separated from the actual interaction and particulate debris/aerosol generated thereby.

While aspects of the present disclosure include examples relating the dental imaging system and/or the dental robotic system to maxillofacial anatomy or maxillofacial structure, a person of ordinary skill in the art will appreciate that reference to the maxillofacial anatomy/maxillofacial structure, in some aspects, is merely to provide the example of an object interacted with/by the disclosed imaging system and/or robotic system. Otherwise, reference herein to an “object” is directed and expressly refers to non-human objects. In some examples, such non-human objects are maxillofacial anatomy models or maxillofacial structure models or other non-human representations or reproductions of such anatomy. The disclosed systems and methods herein are implemented to provide a convenient and effective training tool or training provision for the dental professional to develop their skills in regard to the procedures and tools described herein. Moreover, any methods disclosed and claimed herein are particularly directed to the control and operation of the systems described and claimed herein, wherein such methods are not particularly directed to methods of surgery on humans, but instead to operation of the imaging system/robotic system in relation to the training procedures previously indicated.

Moreover, while aspects of the disclosure illustrate example dental procedures involving maxillofacial anatomy, one skilled in the art will appreciate that the concept of the dental imaging system/dental robotic system disclosed herein may find applicability to other surgical processes not involving dental surgery, such as, for example, orthopedic surgery, ENT surgery, and neurosurgery. As such, the aspects of the disclosure presented herein are merely examples of the applicability of the disclosed concepts and are not intended to be limiting in any manner. That is, aspects of the dental imaging system/dental robotic system disclosed herein may be otherwise applicable to various parts of the patient to facilitate other types of surgery, besides dental surgery.

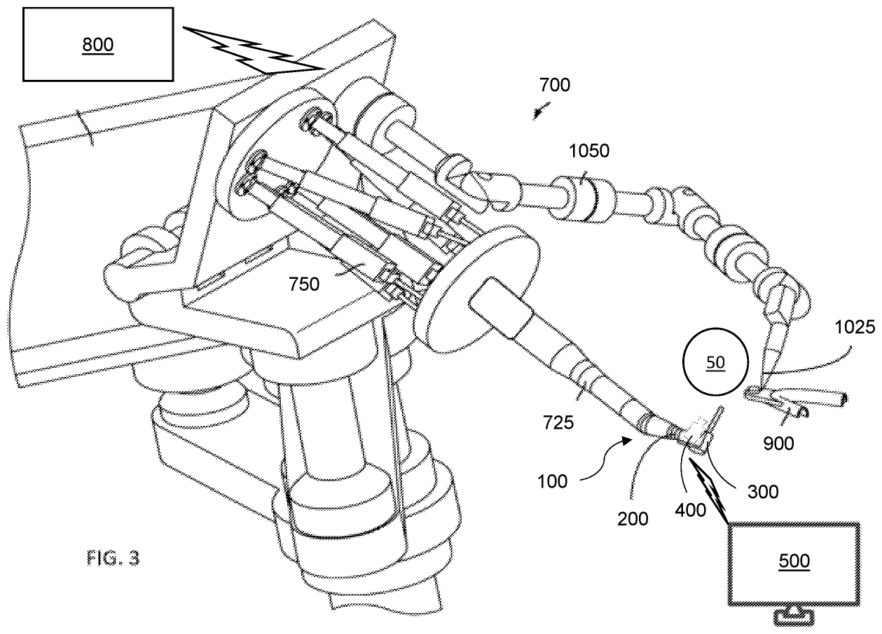

In other aspects, as shown for example in FIG. 3, the dental imaging system 100 is engaged with a dental robotic system 700. That is, in some instances, the dental tool 200 is engaged with the distal end 725 of an articulating arm 750 of the dental robotic system 700, and the end effector 300 of the dental tool 200 is adapted to interact with the object (e.g., maxillofacial structure). The optical imaging device 400 is engaged with the dental tool 200 or the end effector 300 (or in some instances, the distal end 725 of the articulating arm 750), and is arranged such that a field-of-view thereof includes an interaction between the end effector 300 and the object. A controller 800 is arranged in communication with the articulating arm 750, the dental tool 200, and a fiducial marker 900 adapted to engage the object 50.

The controller 800 is arranged, for example, to determine a disposition of the end effector 300 in relation to the fiducial marker 900 during movement of the end effector 300 to interact with the object 50. The controller 800 is further arranged to direct the articulating arm 750 to physically control allowable movement of the dental tool 200, directly relative to the disposition of the end effector 300, with respect to the fiducial marker 900 engaged with the object 50, so as to, for instance, account and adapt for movement of the object 50 during the robotic procedure. For example, in some aspects, the controller 800 is implemented to develop a plan/procedure/or operation which includes the dental tool 200 being directed into proximity with the object 50, as well as the subsequent manipulation of the dental tool 200 to guide the end effector 300 into interaction with the object 50 to perform the procedure. According to aspects of the present disclosure, the developed plan/procedure/operation allows the dental tool 200 to be moved toward and into engagement with the object 50, while the articulating arm 750 (having the dental tool 200 attached to the distal end 725 thereof) includes structure and functionality to allow the dental tool 200 to be moved along an allowable pathway according to the plan/procedure/operation. However, by way of the articulating arm 750, manual movement of the dental tool 200 outside the allowable pathway is restricted, impeded, or otherwise prevented.

The display 500 is in communication with the optical imaging device 400 and is arranged to display a real-time image of the interaction between the end effector 300 and the object 50 received from/captured by the optical imaging device 400. As previously disclosed, the display 500, in particular aspects is disposed remotely to the interaction site between the end effector 300 and the object 50, thereby facilitating safety, convenience, user-friendliness, and ergonomic correctness for the user of the system. The dental imaging system 100 in such implementations with a dental robotic system 700 can thus be configured and arranged according to any of the aspects or any combinations of the aspects as previously addressed herein.

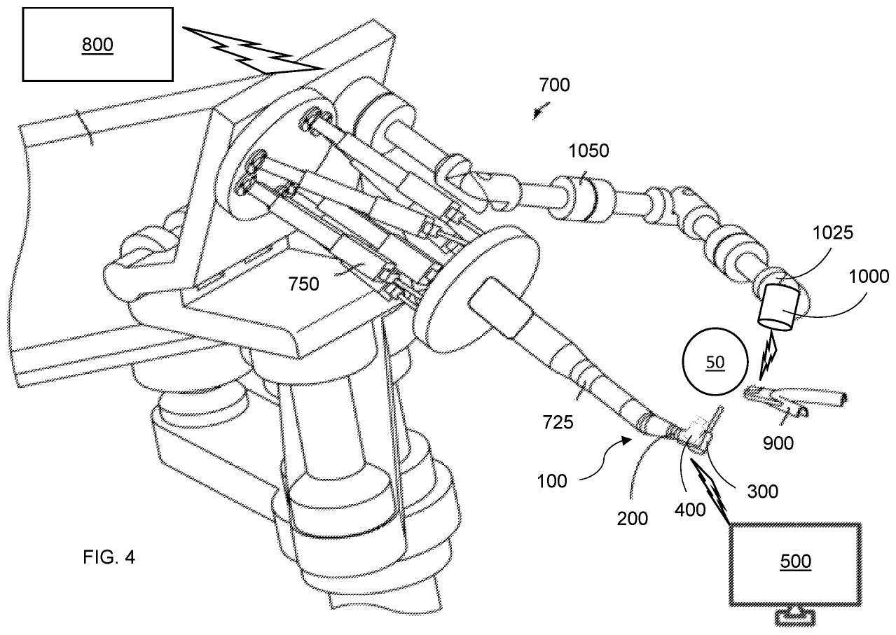

In some aspects (see, e.g., FIG. 3), the distal end 1025 of the tracking arm 1050 is physically engaged with the fiducial marker 900. The tracking arm 1050, in communication with the controller 800, is thus arranged to cooperate with the controller 800 to determine the spatial relationship between the fiducial marker 900 and the end effector 300. In other aspects (see, e.g., FIG. 4), the dental robotic system 700 includes a detector 1000 engaged with a distal end 1025 of a tracking arm 1050, wherein the tracking arm 1050 is a separate and discrete element from the articulating arm 750. The tracking arm 1050 and the detector 1000 are arranged in communication with the controller 800. The detector 1000 is further arranged to cooperate with the tracking arm 1050 to position the detector 1000 in a spaced-apart relationship with the fiducial marker 900, to detect the fiducial marker 900 and to cooperate with the controller 800 to determine a spatial relationship between the fiducial marker 900 and the end effector 300. The detector 1000, in particular example aspects, is an electrical detector, an electromechanical detector, an electromagnetic detector, an optical detector, an infrared detector, or combinations thereof.

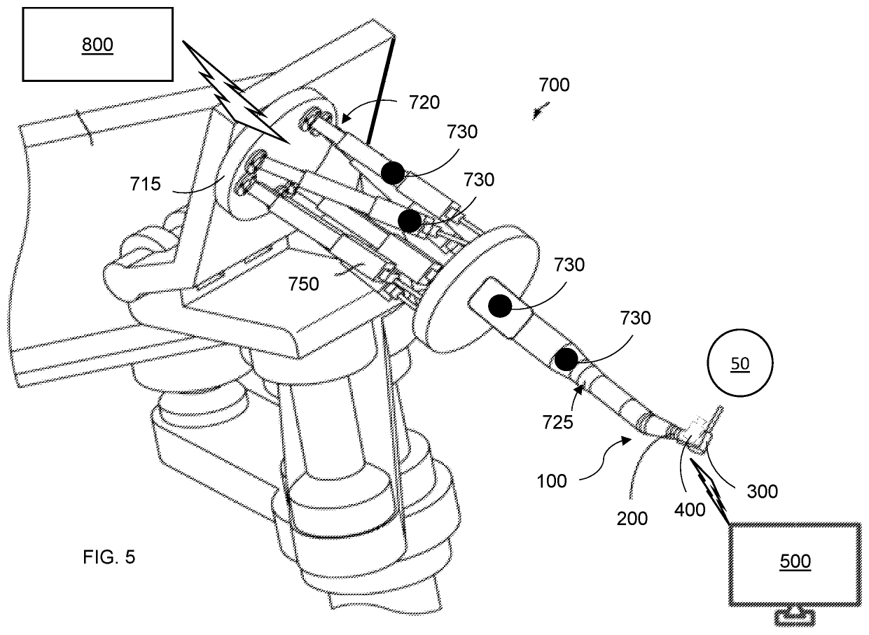

In other aspects, as shown for example in FIG. 5, the dental imaging system 100 is engaged with a dental robotic system 700, comprising an articulating arm 750 having a proximal end 720 and a distal end 725 opposed thereto. One or more sensors 730 is operably engaged with the articulating arm 750 and arranged to sense position data associated with the articulating arm 750. For example, the one or more sensors 730 is engaged with one of the arm members of the articulating arm 750 and/or with a joint engaged between arm members, or between arm members and other components of the articulating arm 750 (e.g., between the proximal end 720 of the articulating arm 750 and a base member 715). In this manner, the position data sensed by the one or more sensors 730 includes, for example, the spatial relationship (e.g., orientation, position, etc.) of the articulating arm 750 and/or the components thereof in a three dimensional space. In some instances, the spatial relationship is determined relative to the base member 715 to which the proximal end 720 of the articulating arm 750 is mounted. As such, in some aspects, the one or more sensors 730 is engaged with the articulating arm 750 such that the position data sensed by the one or more sensors 730 at least indicates the spatial position of at least the distal end 725 of the articulating arm 750 in a three dimensional space, and in some instances relative to the base member 715/proximal end 720 of the articulating arm 750.

FIG. 5 further illustrates an aspect wherein a dental tool 200 is engaged with the distal end 725 of the articulating arm 750 of the dental robotic system 700, and wherein the end effector 300 of the dental tool 200 is adapted to interact with the object (e.g., maxillofacial structure). In such aspects, the optical imaging device 400 engaged with the distal end 725 of the articulating arm 750, the dental tool 200, or the end effector 300, in known relation to the end effector 300. Moreover, the optical imaging device 400 is and arranged such that a field-of-view thereof includes an interaction between the end effector 300 and the object (e.g., the optical imaging device 400 is particularly arranged or directed to capture the interaction between the end effector 300 and the object 50).

The optical imaging device 400 is further arranged to capture imaging data, wherein the imaging data at least includes data regarding an image of the object 50 and/or an image of the interaction between the end effector 300 and the object 50. In this regard, the imaging data for each image captured by the optical imaging device 400 includes or is capable of indicating a corresponding known position of the imaged object in the three-dimensional space based on a calibration of the optical imaging device 400. That is, in some aspects, the optical imaging device 400 is calibrated, for example, in regard to the field of view, the focal length, and/or other parameters indicative of the spatial relation of the imaged object from the imaging element of the optical imaging device 400. Moreover, the position of the optical imaging device 400 (and/or the imaging element thereof) in the three-dimensional space is related to, known from, or otherwise associated with the position of the distal end 725 of the articulating arm 750 determined from the position data of the one or more sensors 730, and/or the known or determined positions of the dental tool 200 and end effector 300 relative to the distal end 725 of the articulating arm 750 (and thus a known or determined relation between the imaged object and the relative positioning of the articulating arm 750. Accordingly, since the spatial relation of the imaged object is known or determinable from the imaging data captured by the optical imaging device 400, the optical imaging device does not necessarily need to be positioned at a particular fixed distance from the imaged object prior to capturing the noted image(s).

A controller 800 is arranged in communication with the articulating arm 750, the one or more sensors 730, the dental tool 200, and the optical imaging device 400. The controller 800 is arranged to receive the position data associated with the articulating arm 750 from the one or more sensors 730, wherein the position data indicates a disposition of the optical imaging device 400 in relation to the proximal end 720 of the articulating arm 750. The controller 800 is further arranged to receive the imaging data associated with an image of the object 50 captured by the optical imaging device 400, wherein the imaging data indicates a disposition of the object 50 in relation to the optical imaging device 400. In such aspects, the controller 800 is arranged to determine a disposition of the end effector 300 in relation to the object 50, from the position data associated with the articulating arm 750 and the imaging data associated with the image, during movement of the end effector 300 to interact with the object 50. From the known disposition of the end effector 300, the controller 800 is further arranged to direct the articulating arm 750 to physically control allowable movement of the dental tool 200, directly relative to the disposition of the end effector 300 with respect to the object 50. A display 500 in communication with the optical imaging device 400, as otherwise disclosed herein, is arranged to display a real-time image of the interaction between the end effector 300 and the object 50 received from the optical imaging device 400.

In particular aspects involving the imaging system 100 and/or the imaging system 100 incorporated into the robotic system 700, the display 500 is arranged to display the real-time image of the interaction between the end effector 300 and the object 50, combined (e.g., by image stitching) with an image of the object 50 not obtained from the optical imaging device 400 (e.g., a CT image, MRI image or other reference image, whether two-dimensional or three-dimensional, used for example for surgical planning purposes), so as to form an augmented virtual representation of the interaction (e.g., a three-dimensional image).

In other aspects, a three-dimensional (3D) reconstructed image (e.g., the augmented virtual representation) can be formed from the two-dimensional (2D) images captured by the optical imaging device 400. This image reconstructions process is facilitated, in particular instances, where the 2D images captured by the optical imaging device 400 are associated with spatial relationship information between the optical imaging device 400 and the object 50 in the 3D coordinate space from the imaging data from the optical imaging device 400, and/or spatial relationship information between the optical imaging device 400 and the articulating arm 750 in the 3D coordinate space from the position data from the one or more sensors 750 engaged with the articulating arm 750. Having the spatial relationship information related with the various 2D images of the object 50 thus facilitates a more accurate and seamless reconstruction of the 3D image (e.g., the augmented virtual representation) of the object 50 and/or the interaction between the end effector 300 and the object 50.

In further aspects, relating the various 2D images of the object 50 with the spatial relationship information associated with the imaging data and/or the position data is additionally augmented by accounting for movement of the object 50 during the imaging process. Consideration of and adjustment for object 50 movement during the imaging process is accomplished, in some instances, by tracking the object by way of the tracking arm 1050/detector 1000 arrangement cooperating with the fiducial marker 900 engaged or otherwise associated with the object 50, or by way of the tracking arm 1050 physically connected to the fiducial marker 900. As such, by utilizing the spatial relationship information associated with the imaging data and/or the position data, with or without the object tracking capability, the imaging data of each image captured by the optical imaging device 400 will be related to the 3D coordinate space relative to the object 50 under consideration. Thus, the plurality of 2D images can be better aligned and stitched more readily and successfully.

In yet further aspects, the facilitate 3D image reconstruction capabilities disclosed herein allow the 3D image reconstruction process to be continually/dynamically updated throughout the interaction of the end effector 300 with the object 50. Such continuous/dynamic updating of the 3D reconstructed image therefore provides a 3D surface model of the object or the interaction between the end effector 300 and the object that is updated in real time or on demand. Such aspects of the present disclosure thus combine the imaging process with known or determined spatial relationships between the optical imaging device 400, the end effector 300 of the dental tool 200, and the object 50 under consideration to facilitate the improved formation of a 3D virtual representation, 3D surface image, or 3D model, while providing capabilities for continuous/dynamic updating of the 3D virtual representation as the interaction between the end effector 300 and the object 50 proceeds through the intended procedure.

Aspects of the present disclosure thus provide, for example, a dental imaging system for facilitating the regulation of the spread of contagions and contaminants by separating the dental professional from the spread of dental aerosols and particulate debris resulting from a maxillofacial procedure. The various aspects of the system further facilitate effective isolation or separation of the dental professional from contagions and contaminants, without limiting the mobility of the dental tool, the accessibility of the dental tool to the maxillofacial structure, or the ability of the dental professional to see the maxillofacial structure without having to be in close proximity thereto. The various aspects of the system further allow the dental professional to conduct and view a maxillofacial procedure in a user-friendly, distanced, and ergonomic manner, while providing a real time, live video feed, in some instances, augmented reality capabilities combined with object (e.g., patient) tracking, and 3D surface image reconstruction for the imaged object or interaction of the end effector therewith. Accordingly, such a system as disclosed is ergonomically friendly for the dental professional, for example, by allowing one-handed operation so as to allow the dental professional to use other instruments (e.g., a dental mirror or suction device) concurrently with using the system.

Many modifications and other embodiments of the inventions set forth herein will come to mind to one skilled in the art to which these disclosed embodiments pertain having the benefit of the teachings presented in the foregoing descriptions and the associated drawings. Therefore, it is to be understood that embodiments of the invention are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the disclosure. In this regard, for example, different combinations of elements and/or functions than those explicitly described above are also contemplated within the scope of the disclosure. Although specific terms are employed herein, they are used in a generic and descriptive sense only and not for purposes of limitation.

It should be understood that although the terms first, second, etc. may be used herein to describe various steps or calculations, these steps or calculations should not be limited by these terms. These terms are only used to distinguish one operation or calculation from another. For example, a first calculation may be termed a second calculation, and, similarly, a second step may be termed a first step, without departing from the scope of this disclosure. As used herein, the term “and/or” and the “/” symbol includes any and all combinations of one or more of the associated listed items.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

That which is claimed:

1. A dental imaging system, comprising:
a dental tool having an end effector adapted to interact with an object;
an optical imaging device engaged with the dental tool or the end effector, and arranged such that a field-of-view thereof includes the interaction between the end effector and the object; and
a display in communication with the optical imaging device and arranged to display a real-time image of the interaction between the end effector and the object received from the optical imaging device.

2. The system of claim 1, wherein the dental tool is a drill and the end effector is a drill bit or an abrading bit, wherein the dental tool is an ultrasonic cleaner and the end effector is a cleaning tip, or wherein the dental tool is a pneumatic polisher and the end effector is a polishing tip.

3. The system of claim 1, wherein the display is mounted on the dental tool, is disposed remotely to the dental tool, or is mounted within a headset adapted to be worn by a user.

4. The system of claim 1, wherein the dental tool is engaged with an articulating arm of a dental robotic system.

5. The system of claim 1, wherein the optical imaging device includes a camera, an imaging array, or an optical fiber disposed adjacent the end effector of the dental tool.

6. The system of claim 5, wherein the end effector defines an axial channel extending toward a distal end of the dental tool, and wherein the optical fiber is disposed within and extends along the axial channel such that a distal end of the optical fiber is disposed proximate the distal end of the dental tool.

7. The system of claim 1, comprising a light-emitting device arranged to illuminate the object or the end effector.

8. The system of claim 1, wherein the optical imaging device is arranged to automatically focus within the field-of-view.

9. The system of claim 1, wherein the optical imaging device is arranged in communication with the display via a wireless communication system.

10. The system of claim 1, wherein the display is arranged to display the real-time image of the interaction between the end effector and the object combined with an image of the object not obtained from the optical imaging device, so as to form an augmented virtual representation of the interaction.

11. A dental robotic system, comprising:
a fiducial marker adapted to engage an object;
an articulating arm having:
a dental tool operably engaged with a distal end thereof and having an end effector adapted to interact with the object, and
an optical imaging device engaged with the dental tool or the end effector, and arranged such that a field-of-view thereof includes an interaction between the end effector and the object;
a controller arranged in communication with the articulating arm, the dental tool, and the fiducial marker, the controller being arranged to:
determine a disposition of the end effector in relation to the fiducial marker during movement of the end effector to interact with the object, and
direct the articulating arm to physically control allowable movement of the dental tool, directly relative to the disposition of the end effector with respect to the fiducial marker engaged with the object; and
a display in communication with the optical imaging device and arranged to display a real-time image of the interaction between the end effector and the object received from the optical imaging device.

12. The system of claim 11, wherein the dental tool is a drill and the end effector is a drill bit or an abrading bit, wherein the dental tool is an ultrasonic cleaner and the end effector is a cleaning tip, or wherein the dental tool is a pneumatic polisher and the end effector is a polishing tip.

13. The system of claim 11, wherein the display is mounted on the dental tool, is disposed remotely to the dental tool, or is mounted within a headset adapted to be worn by a user.

14. The system of claim 11, wherein the optical imaging device includes a camera, an imaging array, or an optical fiber disposed adjacent the end effector of the dental tool.

15. The system of claim 14, wherein the end effector defines an axial channel extending toward a distal end of the dental tool, and wherein the optical fiber is disposed within and extends along the axial channel such that a distal end of the optical fiber is disposed proximate the distal end of the dental tool.

16. The system of claim 11, comprising a light-emitting device arranged to illuminate the object or the end effector.

17. The system of claim 11, wherein the optical imaging device is arranged to automatically focus within the field-of-view.

18. The system of claim 11, further comprising a detector engaged with a distal end of a tracking arm, the tracking arm and the detector being in communication with the controller, the detector being arranged in a spaced-apart relationship with the fiducial marker to detect the fiducial marker and to cooperate with the controller to determine a spatial relationship between the optical imaging device and the fiducial marker or between the end effector and the fiducial marker.

19. The system of claim 18, wherein the detector is an electrical detector, an electromechanical detector, an electromagnetic detector, an optical detector, an infrared detector, or combinations thereof.

20. The system of claim 11, comprising a tracking arm having a distal end physically engaged with the fiducial marker, the tracking arm being in communication with the controller and arranged to cooperate with the controller to determine a spatial relationship between the optical imaging device and the fiducial marker or between the end effector and the fiducial marker.

21. The system of claim 11, wherein the optical imaging device is arranged in communication with the display via a wireless communication system.

22. The system of claim 11, wherein the display is arranged to display the real-time image of the interaction between the end effector and the object combined with an image of the object not obtained from the optical imaging device, so as to form an augmented virtual representation of the interaction.

23. A dental robotic system, comprising:
an articulating arm having a proximal end and a distal end opposed thereto;
one or more sensors operably engaged with the articulating arm and arranged to sense position data associated with the articulating arm;
a dental tool operably engaged with the distal end of the articulating arm and having an end effector adapted to interact with an object;
an optical imaging device engaged with the distal end of the articulating arm, the dental tool, or the end effector, in known relation to the end effector and arranged such that a field-of-view thereof includes an interaction between the end effector and the object, the optical imaging device being further arranged to capture imaging data;
a controller arranged in communication with the articulating arm, the one or more sensors, the dental tool, and the optical imaging device, the controller being arranged to:
receive the position data associated with the articulating arm from the one or more sensors, the position data indicating a disposition of the optical imaging device in relation to the proximal end of the articulating arm,
receive the imaging data associated with an image of the object captured by the optical imaging device, the imaging data indicating a disposition of the object in relation to the optical imaging device,
determine a disposition of the end effector in relation to the object, from the position data associated with the articulating arm and the imaging data associated with the image, during movement of the end effector to interact with the object, and
direct the articulating arm to physically control allowable movement of the dental tool, directly relative to the disposition of the end effector with respect to the object; and
a display in communication with the optical imaging device and arranged to display a real-time image of the interaction between the end effector and the object received from the optical imaging device.

24. The system of claim 23, wherein the dental tool is a drill and the end effector is a drill bit or an abrading bit, wherein the dental tool is an ultrasonic cleaner and the end effector is a cleaning tip, or wherein the dental tool is a pneumatic polisher and the end effector is a polishing tip.

25. The system of claim 23, wherein the display is mounted on the dental tool, is disposed remotely to the dental tool, or is mounted within a headset adapted to be worn by a user.

26. The system of claim 23, wherein the optical imaging device includes a camera, an imaging array, or an optical fiber disposed adjacent the end effector of the dental tool.

27. The system of claim 26, wherein the end effector defines an axial channel extending toward a distal end of the dental tool, and wherein the optical fiber is disposed within and extends along the axial channel such that a distal end of the optical fiber is disposed proximate the distal end of the dental tool.

28. The system of claim 23, comprising a light-emitting device arranged to illuminate the object or the end effector.

29. The system of claim 23, wherein the optical imaging device is arranged to automatically focus within the field-of-view.

30. The system of claim 23, wherein the optical imaging device is arranged in communication with the display via a wireless communication system.

31. The system of claim 23, wherein the display is arranged to display the real-time image of the interaction between the end effector and the object combined with an image of the object not obtained from the optical imaging device, so as to form an augmented virtual representation of the interaction.

32. The system of claim 23, wherein the controller is arranged to associate the position data associated with the articulating arm from the one or more sensors, and the imaging data associated with each of a plurality of two-dimensional images of the object or the interaction between the end effector and the object captured by the optical imaging device.

33. The system of claim 32, wherein the controller is arranged to combine together the plurality of two-dimensional images of the object or the interaction between the end effector and the object, based on the position data and the imaging data, and to form a three-dimensional augmented virtual representation of the object or the interaction between the end effector and the object.

34. The system of claim 23, further comprising a detector engaged with a distal end of a tracking arm, the tracking arm and the detector being in communication with the controller, the detector being arranged in a spaced-apart relationship with the fiducial marker to detect the fiducial marker and to cooperate with the controller to determine a spatial relationship between the optical imaging device and the fiducial marker or between the end effector and the fiducial maker, or further comprising a tracking arm having a distal end physically engaged with the fiducial marker, the tracking arm being in communication with the controller and arranged to cooperate with the controller to determine the spatial relationship between the optical imaging device and the fiducial marker or between the end effector and the fiducial marker.

35. The system of claim 34, wherein the detector is an electrical detector, an electromechanical detector, an electromagnetic detector, an optical detector, an infrared detector, or combinations thereof.

36. The system of claim 34, wherein the controller is arranged to associate the position data associated with the articulating arm from the one or more sensors, the imaging data associated with an image of the object captured by the optical imaging device, and the spatial relationship between the optical imaging device and the fiducial marker with each of a plurality of two-dimensional images of the object or the interaction between the end effector and the object captured by the optical imaging device.

37. The system of claim 36, wherein the controller is arranged to combine the plurality of two-dimensional images of the object or the interaction between the end effector and the object together, based on the position data, the imaging data, and the spatial relationship between the optical imaging device and the fiducial marker, and form a three-dimensional augmented virtual representation of the object or the interaction of the end effector with the object.
